(19) United States
(12) Patent Application Publication
(10) Pub. No.: US 2003/0105027 A1
(43) Pub. Date: Jun. 5, 2003

(54) Nutritional supplements and methods for prevention, reduction and treatment of radiation injury

(76) Inventor: Richard A. Rosenbloom, Elkins Park, PA (US)

Correspondence Address: KNOBLE & YOSHIDA, EIGHT PENN CENTER, SUITE 1350, 1628 JOHN F KENNEDY BLVD, PHILADELPHIA, PA, 19103, US

(21) Appl. No.: 10/132,642

(22) Filed: Apr. 25, 2002

Related U.S. Application Data
(63) Continuation-in-part of application No. 10/045,790, filed on Jan. 14, 2002.
Application No. 10/045,790 is a continuation-in-part of application No. 09/993,003, filed on Nov. 6, 2001.

(51) Int. Cl. A61K 38/05; A61K 31/7048; A61K 31/59; A61K 31/375; A61K 31/353; A61K 35/78; A61K 31/07; A61K 31/355

(52) U.S. Cl. 514/18; 424/94.1; 424/729; 514/168; 514/456; 514/440; 514/474; 514/27; 514/458; 514/725; 514/410

(57) ABSTRACT

A nutritional supplement composition for the prevention, reduction or treatment of radiation injury due to exposure to ionizing radiation, including one or more compounds that regulates cell differentiation and/or cell proliferation, and one or more antioxidants, optionally formulated in a pharmaceutically acceptable carrier for an oral composition. The composition of the present invention may further include optional ingredients such as flavonoids, flavonoid derivatives, selenium, selenium compounds, anti-inflammatories, organic germanium, Korean ginseng, American ginseng, Siberian ginseng and B-complex vitamins. A method for the administration of an oral composition for the purpose of preventing, reducing or treating radiation injury involves orally administering an effective amount of a composition including one or more compounds that regulates cell differentiation and/or cell proliferation, and one or more antioxidants to a person before, during or after radiation exposure. A method for the topical administration of the composition in accordance with the present invention for the purpose of preventing, reducing or treating radiation injury involves topically administering an effective amount of the composition of the invention an area of skin, which has been or will be exposed to ionizing radiation. The compositions and methods can be employed to prevent, reduce or treat radiation injury caused by a wide variety of types of radiation exposure.

### BACKGROUND OF THE INVENTION

1. Field of the Invention

The present invention relates to nutritional supplement compositions and methods for preventing, reducing and treating radiation injury.

2. Description of the Prior Art

It is generally known that extensive radiation exposure or exposure to strong radiation may cause radiation injury. Radiation injury may range from less serious injuries such as radiation dermatitis to more serious injuries such as those causing vomiting, bone marrow failure, intestinal death and/or instant incineration. Such injuries or damage may be caused by radiation emitted from x-rays such as those used in diagnostic equipment, γ-rays such as those emitted from radioactive materials or from numerous other sources.

Many attempts have been made to reduce, control or cure radiation injury. U.S. Pat. No. 5,543,140 to Nakai et al discloses a method of preventing or inhibiting radiation injury by administering interleukin-1-α derivatives. In particular, Nakai et al uses an interleukin-1-α modified by replacing the Asn at the 36 position with Asp, and replacing the Cys at the 141 position with Ser. The modified interleukin-1-α derivative is preferably produced using recombinant DNA techniques, which are complicated and burdensome. In addition, the potential adverse side effects of the modified Interleukin-1-α derivatives are not well known.

U.S. Pat. No. 5,767,092 to Koezuka et al. discloses a composition, which may be therapeutically or prophylactically useful in promoting bone marrow cell proliferation and protecting human bone marrow cells against radiation damage. The composition disclosed in Koezuka et al. contains α-galactosylceramide. However, radiation may cause other injuries in addition to damage to bone marrow cells and thus this composition has limited applicability.

There still remains a need in the art for effective compositions and methods to prevent, reduce and treat radiation injury.

Accordingly, it is an objective of certain embodiments of the present invention to provide an oral composition that, when ingested, will prevent, reduce or treat radiation injury.

It is further objective of certain embodiments of the present invention to provide methods to effectively prevent, reduce or treat radiation injury by oral administration of a composition that, when ingested, will prevent, reduce or treat radiation injury.

It is a still further objective of certain embodiments of the present invention to provide methods of administering a composition to prevent, reduce or treat radiation injury using a combination of oral and topical administration.

These and other objects of the present invention will be apparent from the summary and detailed descriptions of the invention, which follow.

### SUMMARY OF THE INVENTION

In a first aspect, the present invention relates to a nutritional supplement composition for preventing, reducing or treating radiation injury. The nutritional supplement composition includes a compound that regulates cell differentiation and/or cell proliferation, an antioxidant and at least one of a pharmaceutically acceptable carrier for an oral composition or at least one other ingredient useful in the prevention, reduction or treatment of radiation injury.

In a second aspect, the present invention relates to a method of orally administering a composition for the prevention, reduction or treatment of radiation injury. In the method, an effective amount of a suitable composition is orally administered to a person at risk for radiation exposure or to a person who has already been exposed to radiation to prevent, reduce or treat radiation injury.

### DETAILED DESCRIPTION OF THE PREFERRED EMBODIMENTS

In a first aspect, the present invention relates to a nutritional supplement composition for preventing, reducing or treating radiation injury.

The nutritional supplement composition of the present invention includes a compound that regulates cell differentiation and/or cell proliferation, an antioxidant and a pharmaceutically acceptable carrier for an oral composition.

By “nutritional” or “nutritionally-supplemental amount” herein is meant that the supplements used in the practice of this invention provide a nourishing amount of vitamins and minerals. This supplemental amount will comprise at least 3% of the Recommended Dietary Allowance (RDA). Preferably, at least 10% of the RDA will be provided. The RDA for vitamins and minerals is as defined in The United States of America (see Recommended Daily Dietary Allowance—Food and Nutrition Board, National Academy of Sciences—National Research Council). This is supplemental or in addition to the amount found in the diet.

As used herein, the term “flavors” includes both fruit and botanical flavors.

As used herein the term “sweeteners” includes sugars, for example, glucose, sucrose, and fructose. Sugars also include high fructose corn syrup solids, invert sugar, sugar alcohols, including sorbitol, and mixtures thereof. Artificial sweeteners are also included in the term sweetener.

As used herein, a “pharmaceutically acceptable” component is one that is suitable for use with humans and/or animals without undue adverse side effects (such as toxicity, irritation, and allergic response) commensurate with a reasonable benefit/risk ratio. Further, as used herein, the term “safe and effective amount” refers to the quantity of a component which is sufficient to yield a desired therapeutic response without undue adverse side effects (such as toxicity, irritation, or allergic response) commensurate with a reasonable benefit/risk ratio when used in the manner of this invention. The specific “safe and effective amount” will, obviously, vary with such factors as the particular condition being treated, the physical condition of the patient, the duration of the treatment, the nature of concurrent therapy (if any), and the specific formulations employed.

Radiation injury may include injury or damage to any part of the human body caused by exposure to radiation. Such injury or damage may include radiation dermatitis, bone marrow cell damage, intestinal damage, and symptoms or conditions such as cancer, and DNA mutation that may be caused either directly or indirectly, by exposure to one or more ionizing radiations such as fluoroscopic radiation, ultraviolet radiation, proton radiation, alpha radiation, beta radiation, x-ray radiation and gamma radiation. Ionizing radiation can disrupt DNA molecules in living cells and cause mutation, damage, and/or death of the living cells, which in turn may result in cancer and genetic mutation. In addition, ionizing radiation can also cause changes in the chemical balance of cells, which may further cause cancer. However, the term, “radiation injury” as it is used in this application does not include sunburn.

In a preferred embodiment, the compositions and methods of the present invention may be employed to treat radiation injury resulting from exposure to one or more ionizing radiations. Ionizing radiation is any form of radiation that has enough energy to knock electrons out of atoms or molecules, thereby creating ions. Commonly, ionizing radiation includes proton radiation, alpha radiation, beta radiation, x-ray radiation, gamma radiation and neutron radiation. Ionizing radiation may further include cosmic radiation that penetrates the Earth's atmosphere from space and which consists mainly of protons, alpha particles, and heavier atomic nuclei. Positrons, mesons, pions, and other exotic particles can also be found in ionizing radiation. In a more preferred embodiment, the radiation injury being prevented, reduced and/or treated using a method and/or a composition of the present invention is caused by one or more of alpha and beta particle radiation, gamma ray radiation and x-ray radiation.

Alpha and beta particles and gamma rays can come from natural sources or can be technologically produced. Natural radiation comes from cosmic rays, naturally occurring radioactive elements found in the earth's crust (uranium, thorium, etc.), and radioactive decay products such as radon and its subsequent decay products. In addition to these natural sources, radiation can come from such wide-ranging sources as hospitals, research institutions, nuclear reactors and their support facilities, certain manufacturing process, and facilities involved in nuclear weapons production. Radiation can further be a result of a a nuclear power plant accident, a nuclear attack, and/or an accidental nuclear material leakage.

The invention is particularly useful for persons who are, or will be, engaging in activities involving high risk of radiation exposure. Also, the invention can be employed to treat persons exposed to radiation as a result of a radiation attack, a nuclear accident, radiation from diagnostic instruments and therapeutic radiation used to treat, for example, cancer. The radiation injury prevented or treated by the compositions and methods of the present invention may be caused by exposure to non-therapeutic ionizing radiation, such as, for example, accidental radiation exposure, exposure to radioactive materials released by nuclear attack or nuclear accidents, and exposure to diagnostic instruments such as an x-ray machine, a CT-scan, or a synchrotron, all of which employ radiation. Alternatively, the radiation injury prevented or treated by the compositions and methods of the present invention may be caused by exposure to therapeutic radiation, such as radiation therapy used in cancer treatment.

The compound that regulates cell differentiation and/or cell proliferation that may be used in the composition of the present invention may be selected from suitable compounds that have this activity. Suitable compounds that regulate cell differentiation and/or cell proliferation are those that do not induce significant, adverse side effects when administered to a patient in amounts that regulate cell differentiation and/or cell proliferation, and which do not react with one or more of the ingredients of the composition resulting in a substantial loss of activity of one or more of the ingredients. Preferred compounds for regulating cell differentiation and/or cell proliferation are those that occur naturally in the human body and/or materials obtained from plants or animals which may be administered to humans without significant, adverse side effects in the amounts used, or derivatives thereof.

More preferably, the compounds that regulate cell differentiation and/or cell proliferation used in the present invention inhibit or prevent cell differentiation or cell proliferation. Even more preferably, the compounds that regulate cell differentiation and/or cell proliferation used in the present invention accomplish at least one of the following: maintenance of cellular homeostasis and normal cell metabolism, regulation of cell differentiation, induce certain cancer cells to differentiate into normal cells, preferably by working in combination with vitamin A, maintenance of the epidermal permeability barrier, inhibition of cancer cell differentiation, and inhibition of cancer cell proliferation.

Methods for screening compounds that regulate cell differentiation and/or cell proliferation are well-known. For example, DiscoveRx Corporation at Fremont, Calif. markets a Hithunter™ tyrosine kinase assay to detect inhibitors of tyrosine kinase and tyrosine phosphatase which control or regulates cellular growth, proliferation and differentiation using β-galactosidase EFC activity. In this assay, inactive fragments of galactosidase, enzyme acceptor (EA) and enzyme donor (ED) complement to form active enzyme. Binding of an ED-conjugated peptide to an antibody inhibits complementation, while unlabeled peptide displaces the ED-conjugate. This results in increased β-galactosidase activity that is detected subsequently either chemiluminescence or long wavelength fluorescent substrates. Hithunter™ tyrosine kinase assay has been developed to measure activity of the human insulin receptor, EGF receptor kinase domains and Src (EC 50=2.8 nM, 4.4 nM and 4.9 nM respectively). Hithunter™ tyrosine phosphatase activity was also measured using PTP 1B enzyme (EC 50=48 nM). Assay performance characteristics (Z′=0.5-0.7, CV=5-8%) and a simple two step addition protocol make it ideal for HTS (high throughput screening). Another exemplary method for screening compounds that regulate cell differentiation and/or cell proliferation is as available from the Commercial Ventures & Intellectual Property Office at University of Massachusetts, Worcester, Mass. The method can be used to screen for cancer drugs and other drugs that inhibit or promote cell growth, cell death or cell differentiation for diseases involving ERb action, including prostate, breast and ovarian cancer, neurological disorders, osteoporosis and cardiovascular disease. In the method, the effect of any compound on ER-beta regulated cell growth/cell death/cell cycle arrest is determined by adding the compound to culture cells expressing the receptor and measuring alteration in expression levels of ER-beta regulated genes.

Exemplary compounds that regulate cell differentiation and/or cell proliferation are vitamin D₃, vitamin D₃ analogs, compounds that may be converted or metabolized into vitamin D₃ in the human body, and metabolites thereof. Exemplary compounds that may be converted or metabolized into vitamin D₃ include common cholesterols illustrated below. The cholesterol illustrated below may be converted into Provitamin D when a hydrogen is removed from the number 7 carbon, which then forms a double bond with the number 8 carbon, in the second, or ‘B’ ring of the cholesterol molecule. The cholesterol is ‘oxidized’ (that is, an electron is removed with the hydrogen atom), so that the double bond is a consequence of 2 mutually shared electrons between carbons 7 and 8.

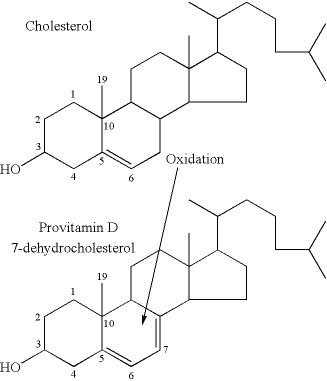

Provitamin D may be converted to Vitamin D₃ by the action of ultraviolet light through human skin. In this reaction, the B ring of the sterol molecule is opened.

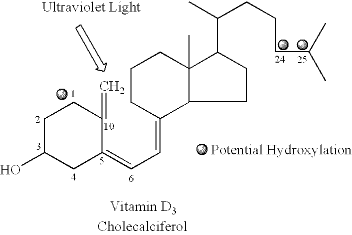

Cholecalciferol, which is Vitamin D₃, may be further converted into another vitamin D intermediate, 25-hydroxycholecalciferol, in the liver by mitochondrial hydroxylase, in the presence of NADPH, and molecular oxygen.

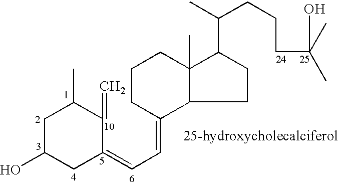

When more active vitamin D₃ is required, 25-hydroxycholecalciferol is transported to the kidney where a new hydrolase enzyme is synthesized. This enzyme introduces another hydroxyl group at position 1, and the bioactive form of Vitamin D₃, calcitriol, is produced.

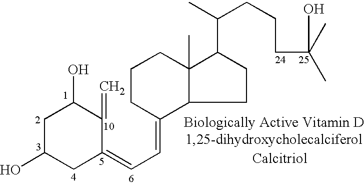

Exemplary vitamin D₃ analogs include I(S), 3(R)-dihydroxy-20(R)-(1-ethoxy-5-ethyl-5-hydroxy-2-heptyn-1-yl)-9,10-seco-pregna-5(Z), 7(E), 10 (19)-triene. Exemplary vitamin D₃ metabolites include 1,25-dihydroxyvitamin D₃. Also, pharmaceutically acceptable salts of the compounds that regulate cell differentiation and/or cell proliferation may be employed. The most preferred compound that regulates cell differentiation and/or cell proliferation is vitamin D₃.

The compound that regulates cell differentiation and/or cell proliferation is used in an amount effective to regulate cell differentiation and/or cell proliferation when orally administered to a patient in the oral composition of the present invention.

Another ingredient in the oral composition of the present invention is the antioxidant. The antioxidant may be a single compound or material or a mixture of two or more compounds and/or materials. Compounds and materials which may be used as antioxidants are those which exhibit antioxidant activity when administered to a patient without causing any severe adverse side affects when used in an amount effective to provide sufficient antioxidant activity, and which do not react with one or more of the ingredients of the composition resulting in a substantial loss of activity of one or more of the ingredients. Preferred antioxidants are those that occur naturally in the human body and/or materials obtained from plants or animals, or derivatives thereof.

Preferred antioxidants are selected from ascorbic acid (vitamin C), its esters, for example, ascorbyl palmitate, and other compounds having vitamin C activity such as those generally called Ester-C® that are disclosed in U.S. Pat. Nos. 4,822,816 and 5,070,085 to Markham, which are incorporated by reference herein; vitamin A and its esters, for example, vitamin A palmitate; vitamin E and its esters, for example, vitamin E acetate; α-lipoic acid, especially DL-α-lipoic acid; carotenoids such as β-carotene; chlorophyllin and its salts; coenzyme Q10; glutathione; green tea polyphenols, such as (−)-epigallocatechin-3-gallate; catechin; galangin; rutin; luteolin; morin; fisetin; silymarin; apigenin; gingkolides; hesperitin; cyaniding; citrin; curcuminoids and structurally similar derivatives thereof which exhibit antioxidant activity. Even more preferably, mixtures of two or more antioxidants are employed in the composition of the present invention. Particularly preferred antioxidant mixtures are ascorbyl palmitate with one or more of vitamin A, vitamin E acetate and α-lipoic acid especially DL-α-lipoic acid. The antioxidants may also be used in the form of their pharmaceutically acceptable salts and this may be preferred in some cases to increase solubility or dispersability, to reduce adverse side effects, etc. In another particularly preferred embodiment, the antioxidants used in the composition of the present invention includes at least one of the compositions having vitamin C activity disclosed in U.S. Pat. Nos. 4,822,816 and 5,070,085 to Markham, which are also commonly called Ester-C®. The Ester-C® disclosed in U.S. Pat. Nos. 4,822,816 and 5,070,085 to Markham generally includes (a) an effective amount of a compound having Vitamin C activity. As used herein, the term, “compound having Vitamin C activity” means Vitamin C (L-ascorbic acid) and any derivatives thereof which exhibit antiscorbutic activity. Such derivatives include, for example, oxidation products, such as dehydroascorbic acid and edible salts of ascorbic acid such as, illustratively, calcium, sodium, magnesium, potassium and zinc ascorbates, esters of Vitamin C with organic and inorganic acids, such as L-ascorbic acid 2-0-sulfate, L-ascorbic acid 2-0-phosphate, L-ascorbic acid 3-0-phosphate, L-ascorbic acid 6-hexadecanoate, L-ascorbic acid monostearate, L-ascorbic acid dipalmitate and the like.

Metabolites of ascorbic acid and its derivatives include the aldonic acids, aldono-lactones, aldono-lactides and edible salts of aldonic acids. Preferably, the compound having Vitamin C activity includes one or more of these metabolites selected from L-threonic acid, L-xylonic acid and L-lyxonic acid. The presence of one or more of these metabolites in the compositions of the invention may provide an improvement in absorption and/or retention of Vitamin C or other therapeutically active compounds.

Structurally similar derivatives of one or more of these compounds, which exhibit antioxidant activity when administered in the oral compositions of the present invention, may also be employed. By “structurally similar derivatives” is meant derivatives that exhibit antioxidant activity and contain at least one significant, common structural element with the compound or material from which it is derived.

In another preferred embodiment, the antioxidant used in the composition of the present invention may include one or more antioxidant enzymes. The antioxidant enzymes useful in the present invention are those capable of scavenging radicals, promoting radical scavengers or preventing radical formation. The preferred antioxidant enzymes useful in the present invention include superoxide dismutase, catalase, glutathione peroxidase and methionine reductase. Other antioxidant enzymes with activities similar to those mentioned explicitly above, may also be used. In addition, one or more of the antioxidant enzymes may act in combination with one or more of the antioxidant compounds in the composition to, for example, scavenge free radicals and/or prevent cell damage in the skin.

The antioxidant component of the composition is used in an amount effective to provide significant antioxidant activity when administered to a patient in the composition of the present invention.

The ratio of the amount of the compound that regulates cell differentiation and/or cell proliferation to the amount of antioxidant employed in the compositions of the present invention is from about 200 IU per gram of antioxidant to about 3 million IU per gram of antioxidant. More preferably, the ratio of the amount of the compound that regulates cell differentiation and/or cell proliferation to the amount of antioxidant employed in the compositions of the present invention is from about 1800 IU per gram of antioxidant to about 1 million IU per gram of antioxidant, and, most preferably the ratio is from about 5000 IU per gram of antioxidant to about 200,000 IU per gram of antioxidant.

The antioxidants used in the composition of the present invention are preferably selected not only for their antioxidant activity, but also based on other beneficial effects that particular compounds may provide. For example, a racemic mixture of α-lipoic acid not only has a strong antioxidant activity but also has a recycling effect on vitamins C and E, and thus is a particularly preferred antioxidant for the present invention. In addition, α-lipoic acid can function in both lipid and non-lipid environments. Similarly, vitamin E and its esters may contribute to an anti-cancer effect and may have beneficial effects on the skin and is thus is also a preferred antioxidant. Vitamin C and its esters are not only antioxidants, but also exhibit a strong combinatorial effect with vitamin E and its esters when used together. In fact, vitamin E and its esters, and vitamin C and its esters can mutually reinforce one another by a mechanism in which one antioxidant (reducing agent) acts as a regenerator for the oxidized form of the other. In addition, some of the antioxidants useful in the present invention are more active in a lipid environment whereas others are more active in a non-lipid environment. Accordingly, the composition of the present invention may preferably include a combination of at least two antioxidants, with one being selected for its higher activity in a lipid environment and a second one being selected for its higher activity in a non-lipid environment.

Vitamin A (retinol or retinyl ester) may also have anti-cancer effects. In addition, vitamin A may also enhance the physiological mechanism of cell differentiation, inhibit malignant transformation, suppress tumor promotion and directly act against neoplastic cells. Vitamin A is also a fat-soluble material and thus is preferred for use due to this additional beneficial property. Preferably, vitamin A may be used in its ester forms, such as vitamin A palmitate, because the ester forms of vitamin A may be less irritating to the stomach.

Another particularly preferred antioxidant is green tea polyphenol or green tea extract, which contains compounds such as (−)-epigallocatechin-3-gallate, (−)-epigallocatechin-3-gallate, (−)-epigallocatechin and/or (−)-epicatechin. Studies (see Elmets, C. A. et al, J. Am. Acad. Dermatol., 44 (3); 425-32, March, 2001) have shown that green tea polyphenol or extract is effective in inhibiting erythema and preventing Langerjans cells from some forms of ultraviolet radiation damage.

Carotenoids such as β-carotene may also be included in the composition of the present invention as a preferred antioxidant. Several carotenoids have shown beneficial effects for the present application, such as enhancement of immune response, inhibition of mutagenesis and/or reduction of induced nuclear damage. Carotenoids can also protect against photo-induced tissue damage. Some carotenoids, including β-carotene, quench highly reactive singlet oxygen under certain conditions and can block free radical-mediated reactions.

Preferably, the antioxidant used in the composition of the present invention may also include one or more curcuminoids. Exemplary curcuminoids include curcumin (diferuloylmethane), desmethoxycurcumin (hydroxycinnamoyl feruloylmethane), and/or bis-desmethoxycurcumin (dihydroxydicinnamoyl methane) (see Drug Analysis by Chromatography and Microscopy, p. 169, Ann Arbor Science Inc., 1973), which may be purchased from commercial sources or isolated from turmeric. Methods for isolating curcuminoids from turmeric are known, (see Janaki and Bose, An Improved Method for the Isolation of Curcumin From Turmeric, J. Indian Chem. Soc. 44:985 (1967)). Alternatively, curcuminoids for use in the present invention can be prepared by synthetic methods. Curcumin not only has antioxidant properties but also may have anti-inflammatory, anti-tumor and other valuable properties.

Preferably, the antioxidant used in the composition of the present invention may also include chlorophyllin and/or its salts, because chlorophyllin and its salts may exhibit beneficial effects such as an anti-cancer effect, protection of DNA against ionizing radiation and other chemical mutagens, and fighting bad breath, nausea and indigestion, in addition to being a potent antioxidant. Chlorophyllin and its salts may be included in the composition of the present invention as part of the antioxidant. More preferably, chlorophyllin and its salts may be included in the composition of the present invention in amounts, which, when administered to a patient according to a method of the present invention, provide a daily dosage between about 20 milligrams and about 500 milligrams. Chlorophyllin and its salts may be an alfalfa extract or extracted from silkworm feces. Chlorophyllin and its salts may also be purchased from common commercial sources such as Aldrich Chemical Company.

Even more preferably, the antioxidant used in the composition of the present invention includes a combination of effective amounts of vitamin A or its esters, vitamin C or its esters, vitamin E and α-lipoic acid to achieve the beneficial effect of recycling vitamin C or its esters and vitamin E by α-lipoic acid.

Preferably, the composition of the present invention further includes one or more flavonoids and/or flavonoid derivatives. These flavanoids and/or flavanoid derivatives may have radioprotective effects. In addition, flavonoids and/or flavonoid derivatives such as quercetin may have other beneficial effects such as acting as an anti-inflammatory and maintaining the structural integrity of ischemic or hypoxic tissue, which may occur after radiation exposure. Exemplary flavonoids and flavonoid derivatives include 1,2,3,6-tetra-o-gallyol-β-d-glucose; 2′o-acetylacetoside; 3,3′,4-tri-o-methyl-ellagic acid; 6,3′,4′-trihydroxy-5,7,8-trimethoxyflavone; 6-hydroxy-luteolin; 6-hydroxykaempferol-3,6-dimethyl ether; 7-o-acetyl-8-epi-loganic acid; acacetin; acetoside; acetyl trisulfate quercetin; amentoflavone; apigenin; apiin; astragalin; avicularin; axillarin; baicalein; brazilin; brevifolin carboxylic acid; caryophyllene; chrysin-5,7-dihydroxyflavone; chrysoeriol; chrysosplenol; chrysosplenoside-a; chrysosplenoside-d; cosmosiin; δ-cadinene; dimethylmussaenoside; diacerylcirsimaritin; diosmetin; dosmetin; ellagic acid; ebinin; ethyl brevifolin carboxylate; flavocannibiside; flavosativaside; genistein; gossypetin-8-glucoside; haematoxylin; hesperidine; hispiduloside; hyperin; indole; iridine; isoliquiritigenin; isoliquiritin; isoquercitrin; jionoside; juglanin; kaempferol-3-rhamnoside; kaempferol-3-neohesperidoside; kolaviron; licuraside; linariin; linarin; lonicerin; luteolin; luetolin-7-glucoside; luteolin-7-glucoside; luetolin-7-glucoronide; macrocarpal-a; macrocarpal-b; macrocarpal-d; macrocarpal-g; maniflavone; methy scutellarein; naringenin; naringin; nelumboside; nepetin; nepetrin; nerolidol; oxyayanin-a; pectolinarigenin; pectolinarin; quercetagetin; quercetin; quercimertrin; quercitrin; quercitryl-2″acetate; reynoutrin; rhamnetin; rhoifolin; rutin; scutellarein; sideritoflavone; sophoricoside; sorbarin; spiraeoside; trifolin; vitexin; and wogonin.

The most preferred flavonoids and/or flavonoid derivatives are quercetin, quercetrin, myricetin, kaempferol and myrecetrin since these compounds may have some anti-inflammatory activity and/or may help stabilize cell membranes in combination with a relatively low toxicity, both of which activities may be beneficial in the treatment of radiation. Also, pharmaceutically acceptable salts of these flavonoids and/or flavonoid derivatives may be employed. The particular flavonoids and/or flavonoid derivative included in the composition may be determined by factors such as toxicity, bioavailability, solubility or dispersability, among others.

The particular flavonoids and/or flavonoid derivatives mentioned above are also preferred since some of these compounds may provide additional beneficial effects in the composition of the present invention. For example, quercetin may also have an antioxidative and anticlastogentic effect. It may prevent the decrease of endogenous ascorbic acid (vitamin C) in bone marrow after gamma-ray irradiation. In addition, some of the flavonoids and flavonoid derivatives may act as a radical scavenger to scavenge free radicals such as hydroxyl radicals to enhance their radioprotective effects.

In a more preferred embodiment, both quercetin and ascorbyl palmitate are included in the composition of the present invention because there seems to be an enhanced antioxidant effect of the combination of quercetin and ascorbyl palmitate.

The flavonoids and/or flavonoid derivatives are used in an amount of about 0.02 to about 2 grams per gram of the total antioxidant in the composition. More preferably, the flavonoids and/or flavonoid derivatives are employed in an amount of about 0.05 to about 1 gram, per gram of the total antioxidant in the composition, and, most preferably, the flavanoids and/or flavanoid derivatives are employed in an amount of 0.1 to about 0.4 grams per gram of the total antioxidant in the composition.

The composition of the present invention may further include selenium and/or a compound containing selenium. Selenium is known to be able to prolong the lifespan of a person exposed a severe dose of harmful radiation, e.g. as a result of the Chernobyl accident, and to reduce the potential occurrence of leukemia and other malignancies in that person. Selenium may be included in the composition of the present invention in such an amount that when the composition is administered to a human according to a method of the present invention, the daily dosage should be between 5 micrograms and 200 micrograms. Preferably, selenium may be included in the composition in such an amount that when the composition is administered to a human according to a method of the present invention, the daily dosage should be between 10 micrograms and 100 micrograms. An excessive amount of selenium and/or selenium compound in the composition of the present invention may render the composition toxic.

The oral and/or topical compositions of the present invention may further include an organic germanium compound such as carboxy ethyl sesquioxide of germanium or spirogermanuium. Organic germaniums are known to protect human cells from radiation damage. For example, controlled experiments have also shown that Ge-132 reduces mutations in E. coli due to γ-radiation by twenty-fold (see Mochizuki and Kada, Antimutagenic effect of Ge-132 on γ-ray-induced mutations in E. coli B/r WP2 trp- 42(6) Int. J. Radiat. Biol, 653-59 (1982)). Germanium oxide has been shown to reduce the mutation rate in Salmonella typhimurium induced by Trp-P-2(3-amino-1-methyl-5H-pyrido(4,3-b)indole), by 40-67 folds (see Kada, Mochizuki, and Miyao, Antimutagenic Effects of Germanium Oxide on Trp-P-2 Induced Frameshift Mutations in Salmonella Typhimurium TA98 and TA 1538, 125 Mutation Research, 145-51 (1984)). One or more organic germaniums may be included in the composition of the present invention in such an amount that when the composition is administered to a human according to a method of the present invention, the daily dosage of the germanium compound will be between 25 milligrams and 500 milligrams. Preferably, the organic germanium may be included in the composition in such an amount that when the composition is administered to a human according to a method of the present invention, the daily dosage of the germanium compound will be between 50 milligrams and 200 milligrams, and, most preferably, about 100 milligrams.

Alternatively, Siberian ginseng may be added to the oral and/or topical compositions of the present invention in the form of one or more of Siberian ginseng roots, Siberian ginseng powder, or extracts thereof which may contain one or more of the active ingredients of the Siberian ginseng. Siberian ginseng (Eleutherococcus senticosus) has been shown to have restorative effects on the functions of bone marrow damaged by exposure to radiation. The active ingredients of Siberian ginseng generally include eleutherosides A, B, B1, C, D and E; triterpenoid saponins;

eleutherans A, B, C, D, E, F and G; and equivalents thereof. Siberian ginseng extract may be included in the composition of the present invention in such an amount that when the composition is administered to a human according to a method of the present invention, the daily dosage of the Siberian ginseng extract will be between 25 milligrams and 500 milligrams. Preferably, Siberian ginseng extract may be included in the composition in such an amount that when the composition is administered to a human according to a method of the present invention, the daily dosage of Siberian ginseng extract should be between 50 milligrams and 150 milligrams, and, most preferably, the daily dosage of the Siberian ginseng extract will be about 100 milligrams. If Siberian ginseng is used in a different form in the composition of the present invention, a skilled person should be able to adjust the amount being used accordingly based on the dosages for the Siberian ginseng extract given above.

Alternatively, the compositions of the present invention may include Korean ginseng (panax ginseng) and/or American ginseng (panax quinquefolius), in the form of roots, powder or an extract. Korean and/or American ginseng may prompt recovery of hemateikon and splenal weight and cause improvement of thrombocyte cells. This product is commercially available as Korea Insam. The daily dosage for Korean and/or American ginseng is the same as for Siberian ginseng. A skilled person is able to adjust the dosage of the Korean and/or American ginseng for different physical forms of administration, i.e. root, powder or extract. Of course, mixtures of one or more of Siberian ginseng, Korean ginseng and American ginseng and/or extracts of one or more of these ginseng types may also be employed.

Particularly preferred compositions in accordance with the present invention contain 3,800-4,800 IU of vitamin A palmitate; 2,400-7,200 IU of beta carotene; 240-480 IU vitamin D₃; 95-300 IU of vitamin E in the form of alpha-tocopherol; 48-72 mg of alpha-lipoic acid; 280-580 mg of quercetin, 120-240 mg of ascorbyl palmitate; 4.5-7.2 mg of curcumin; 4.5-10 mg of green tea (C&P); 45-100 mg of chlorophyllin; 24-100 mg of carboxy ethyl sesquioxide of germanium and 180-540 mcg of superoxide dismutase for every gram of non-carrier ingredients contained therein, wherein the non-carrier ingredients may include the compound that regulates cell differentiation and/or proliferation, the antioxidant, preferably, the flavonoids and/or flavonoid derivatives, and optionally selenium, organic germaniums and Siberian ginseng.

The composition in accordance with the present invention may provide one or more of the following beneficial effects to a patient when orally administered in an effective amount: antioxidant properties, free radical scavenging, transition metal chelation, nitric oxide stabilization, anti-inflammatory activity, relief of pain, burning, tingling, electrical sensations and/or hyperalgesia, increased microcirculation, nitric oxide stabilization, promotion of healing of skin ulcers and lesions, protein kinase C inhibition, decreased oxidative stress, anti-inflammation, protection against radiation damage, blockage of the formation of leukotrienes, stabilization of cell membranes, and regulation of cell differentiation, cell proliferation protection of mitochondrial membranes, reduction of cell damage, especially damage to DNA molecules, and plays a role in the repair and regeneration process of damages cells.

In one preferred embodiment, the nutritional supplement compositions of the present invention may be formulated in any acceptable oral dosage forms including, but not limited to, capsules, tablets, lozenges, troches, hard candies, powders, sprays, elixirs, syrups, and suspensions or solutions.

The oral compositions of the present invention are preferably formulated with a pharmaceutically acceptable carrier. The pharmaceutically acceptable oral carrier may include, but is not limited to: (a) carbohydrates including fructose, sucrose, sugar, dextrose, starch, lactose, maltose, maltodextrins, corn syrup solids, honey solids, commercial tablet compositions including Emdex.RTM., Mor-Rex.RTM., Royal-T.RTM., Di-Pac.RTM., Sugar-Tab.RTM., Sweet-Rex.RTM., New-Tab.RTM., (b) sugar alcohols including mannitol, sorbitol, xylitol, and (c) various relatively insoluble excipients including dicalcium phosphate, calcium sulfate, calcium carbonate, microcrystalline cellulose and other pharmaceutical tableting ingredients.

In the case of tablets, for oral use, the pharmaceutically acceptable oral carrier may further include lactose and corn starch. Lubricating agents may also be added to the tablets, including, for example, magnesium stearate, sodium lauryl sulfate and talc. Tablets may also contain excipients such as sodium citrate, calcium carbonate and calcium phosphate. Disintegrants such as starch, alginic acid and complex silicates, may also be employed. Tablets may also include binding agents such as polyvinylpyrrolidone, gelatin, PEG-8000 and gum acacia.

In the case of lozenges for oral use, the common pharmaceutically acceptable oral carrier may further include a binder such as PEG-8000. Preferably lozenges are made in a 0.1 to 15 grams size to allow a suitable dissolution rate for lozenges. More preferably lozenges are made in a 1 to 6 gram size to allow a suitable dissolution rate for lozenges. Dissolution time should be about 15 minutes in water bath testers at 37° C. degrees or about 30 minutes when orally dissolved as lozenges for treating a sore throat, congestion, laryngitis and mucous membrane inflammation.

To directly make compressible lozenges, add the active ingredients to PEG-8000 processed fructose; or add the active ingredient of the composition to crystalline fructose and commercially available, sweet, direct compression products such as Mendell's Sugartab.RTM., Sweetrex.RTM., or Emdex.RTM and add saccharin if desired, flavors as desired, glidants such as silica gel as needed, and lubricants such as magnesium stearate, as needed. The mixture should be kept dry and tableted soon after mixing. The ingredients are mixed and directly compressed into lozenges using conventional pharmaceutical mixing and tableting equipment. The compressive force is preferably sufficient to produce maximum hardness throughout the lozenges, to preserve the dissolution rate, and to maximize the efficacy of lozenges. Dissolution should occur over a sustained period of time of about 5 to 60 minutes, and preferably about 20 to 30 minutes. The composition should be stored in an airtight container and in a cool dark place. Tablets and troches can be manufactured using procedures similar to that described above with minor changes in the optional ingredients.

Alternatively, the oral composition of the present invention may be formulated in liquid form, such as syrups, mouthwashes or sprays with a solvent or dispersant such as water, or other liquids in a pharmaceutically acceptable oral carrier for delivery of the composition to a patient.

The oral composition may also be formulated in chewable compositions such as soft candy, gum drops, liquid filled candies, chewing gum bases and dental supplies, such as toothpastes and mouthwashes by further including fructose, sucrose, or saccharin in the composition, as needed.

The oral composition of the invention may be formulated in capsule form with or without diluents. For capsules, useful diluents include lactose and dried cornstarch. When suspensions are employed, emulsifying and/or suspending agents may be employed in the suspensions. In addition, solid compositions including one or more of the ingredients of the lozenges described above may be employed in soft and hard gelatin capsules.

The compositions of the present invention may also be formulated into a nasal aerosol or inhalant. Such compositions may be prepared using well-known techniques. For these types of formulations, suitable carriers may include the following ingredients: saline with one or more preservatives, absorption promoters to enhance bioavailability, fluorocarbons and/or other conventional solubilizing or dispersion agents.

Other materials, which may optionally be included in the oral composition of the present invention, include inositol, other B-complex vitamins, preservatives, emulsifying agents, suspending agents, melting agents, excipients, and solvents or and anti-inflammatories. Also, ingredients such as sweeteners, flavorants, coloring agents, dyes, and diluents such as water, ethanol, propylene glycol, glycerin and various combinations thereof, may be included in the oral composition of the present invention.

The optional sweeteners which may be used in the oral composition of the present invention include, but are not limited to, saccharin, aspartame, cyclamates, acesulfame K, neohesperidin dihydrochalcone, other super sweeteners, and mixtures thereof, which may be added to the carrier in amounts sufficiently low so as not to chemically interact with the non-carrier ingredients of the oral composition.

The optional flavorants which may be used in the oral composition of the present invention include, but are not limited to, peppermint, peppermint-menthol, eucalyptol, wintergreen, licorice, clove, cinnamon, spearmint, cherry, lemon, orange, lime, menthol and various combinations thereof.

In general, the non-carrier ingredients described above, which may include the compound that regulates cell differentiation and/or proliferation, the antioxidant, preferably, the flavonoids and/or flavonoid derivatives, and optionally selenium, organic germanium, Korean ginseng, American ginseng and Siberian ginseng make up from about 0.5-50% by weight of the total composition. Preferably, the non-carrier ingredients will make up about 1-20% by weight of the total composition. More preferably, the non-carrier ingredients make up about 2-10% by weight of the total composition.

In a second preferred embodiment, the nutritional supplement composition of the present invention is an oral composition, which includes a mixture of a compound that regulates cell differentiation and/or cell proliferation, an antioxidant and at least one other ingredient useful in the prevention, reduction or treatment of radiation injury. The at least one additional ingredient may be selected from flavonoids and/or flavonoid derivatives, selenium and/or selenium compounds, inositol, other B-complex vitamins, organic germanium, Korean ginseng, American ginseng, Siberian ginseng, extracts of one or more of these ginseng types and anti-inflammatories. These ingredients may be employed in the same relative amounts as given above.

The nutritional supplement composition of the present invention may be administered to a person in any orally acceptable dosage form including, but not limited to tablets, capsules, lozenges, troches, hard candies, powders, gels, sprays, elixirs, syrups, and suspensions, solutions, mouthwashes, sprays with a solvent or dispersant such as water, or other liquids in a pharmaceutically acceptable oral carrier for delivery of the composition to a person. The composition may also be formulated in chewable compositions such as soft candy, gum drops, liquid filled candies, chewing gum bases and dental supplies, such as toothpastes and mouthwashes by further including fructose, sucrose, or saccharin in the composition, as needed.

The nutritional supplement composition may be administered 1-10 times per day, as needed, more preferably, 2-6 times per day, as needed, or most preferably, 3 times per day, to a person. Preferably, during each administration of a dose, 1-5 tablets, capsules, lozenges, or equivalents thereof, are ingested by the person. More preferably, 1-2 tablets, capsules, lozenges or equivalents thereof are ingested by the person during each administration of a dose. Most preferably, the tablets, capsules or lozenges or equivalents thereof are ingested with a fluid such as water, juice, milk, or other suitable fluids.

The effective amount of the nutritional supplement will vary depending on such factors as the patient being treated, the particular mode of administration, the activity of the particular active ingredients employed, the age, bodyweight, general health, sex and diet of the patient, time of administration, rate of excretion, the particular combination of ingredients employed, the total content of the main ingredient of the nutritional supplement, and the severity of the illness or symptom. It is within the skill of the person of ordinary skill in the art to account for these factors.

In another aspect, the present invention relates to a method of preventing, reducing or treating radiation injury by the oral administration of an amount of a composition, which includes a mixture of a compound that regulates cell differentiation and/or cell proliferation, and at least one antioxidant, which is effective to prevent, reduce or treat radiation injury.

In the preferred embodiment, the method of the present invention involves the oral administration of a composition to a human that may be potentially exposed to ionizing radiation, is in the process of being exposed to ionizing radiation, or has already been exposed to ionizing radiation. Preferably, the ionizing radiation is selected from alpha-radiation, beta-radiation, gamma ray radiation, and x-ray radiation. The effective amount of the oral composition to be administered will vary depending on such factors as the person being treated, the particular mode of administration, the activity of the particular non-carrier ingredients employed, the age, bodyweight, general health, sex and diet of the person, the time of administration, the rate of excretion, the particular combination of ingredients employed, the total content of the non-carrier ingredients of the oral composition, and the severity of the radiation injury or expected radiation exposure. It is within the skill of the person of ordinary skill in the art to account for these factors to provide a suitable dosage and treatment regimen for a standard 70 kg adult, described below.

As discussed above, the oral composition of the present invention may be administered to a patient in any orally acceptable dosage form including, but not limited to tablets, capsules, lozenges, troches, hard candies, powders, sprays, elixirs, syrups, and suspensions, solutions, mouthwashes, sprays with a solvent or dispersant such as water, or other liquids in a pharmaceutically acceptable oral carrier for delivery of the composition to a patient. The oral composition may also be formulated in chewable compositions such as soft candy, gum drops, liquid filled candies, chewing gum bases and dental supplies, such as toothpastes and mouthwashes by further including fructose, sucrose, or saccharin in the composition, as needed. The compositions of the present invention may also be formulated into a nasal aerosol or inhalant.

The oral composition may be administered 1-10 times per day, as needed, more preferably, 2-6 times per day, as needed, or most preferably, 3 times per day, to a person before, during and/or after radiation exposure. Preferably, during each administration of a dose, 1-5 tablets, capsules, lozenges, or equivalents thereof, are ingested by the person. More preferably, 1-2 tablets, capsules, lozenges or equivalents thereof are ingested by the person during each administration of a dose. Most preferably, the tablets, capsules or lozenges or equivalents thereof are ingested with a fluid such as water, juice, milk, or other suitable fluids.

Preferably, an effective amount of the composition for each administration contains 0.1 gram to 1 gram of the non-carrier ingredients, including, but not limited to, a compound that regulates cell differentiation and/or cell proliferation, and an antioxidant. Preferably, one or more of the flavonoids and/or flavonoid derivatives and/or at least one of selenium and selenium compounds are also included in the composition for oral administration as non-carrier ingredients. More preferably, an effective amount of the composition for each administration contains 0.2 gram to 0.5 gram of the non-carrier ingredients.

In a more preferred embodiment, the method of the present invention further includes the step of topically applying a composition which includes a mixture of a compound that regulates cell differentiation and/or cell proliferation, an antioxidant and a pharmaceutically acceptable topical carrier to an area of the skin prior to, during or after exposure of that area of skin to radiation. In the method, an effective amount of the topical composition of the invention is applied to the skin one to six times daily, as needed.

For prevention or reduction of radiation injury, the topical composition is preferably applied to the skin before potential exposure to radiation. More preferably, the topical composition of the present invention is applied to the skin at least once twenty-four hours before the start of the potential radiation exposure, and three times (e.g., morning, noon and bedtime) in the 24-hour period immediately before the potential radiation exposure. For each application, it is preferable to apply an amount of the composition, which is sufficient to cover the area of the skin to be potentially exposed to radiation with a thin layer of the topical composition. The topical composition should preferably be rubbed into the skin until little or no residue remains on the skin.

In a method for treating or reducing radiation injury, an effective amount of the topical composition of the invention is applied one to six times daily, as needed, to an area of skin inflicted with radiation injury during and/or after radiation exposure. In the method, a thin layer of the topical composition is preferably applied to the inflicted area of skin, as needed, and the topical composition should preferably be rubbed into the skin until little or no residue remains on the skin.

The method of the present invention, which employs combined oral and topical administration may provide one or more of the beneficial effects described above for the compositions of the invention. In addition, the method of the present invention may provide one or more additional beneficial effects due to one or more of the ingredients contained in the pharmaceutically acceptable oral or topical carriers as described above.

The pharmaceutically acceptable topical carrier used in the present invention may be a carrier suitable for use as a carrier for topical compositions. The non-carrier ingredients, which may include a compound that regulates cell differentiation and/or cell proliferation, an antioxidant, and optionally one or more flavonoids and/or flavonoid derivatives, selenium and /or a selenium compound, as well as inositol, other B-complex vitamins, and anti-inflammatories such as y-linolenic acid, are dissolved, dispersed and/or suspended in the topical composition. Exemplary topical carriers may include creams, ointments, lotions, pastes, jellies, sprays, aerosols, bath oils, and other topical pharmaceutical carriers, which accomplish direct contact between the active ingredients of the topical composition of the present invention and the pore of the skin. Preferably, the pharmaceutically acceptable topical carrier may make up more than about 80%, and more preferably about 80-95% w/w of the total composition. In some cases, it may be necessary to dissolve one or more the active ingredients in an appropriate solvent such as ethanol or DMSO (dimethylsulfoxide), and the like, to facilitate the incorporation of the one or more active ingredients into the topical composition or the pharmaceutically acceptable topical carrier.

One preferred topical carrier useful in the present invention may contain at least a hydrophilic ointment base, panthenol or a panthenol derivative and a dispersant if needed to disperse one or more insoluble or partially insoluble active ingredients in the carrier. Another preferred topical carrier of the present invention employs hydroxymethyl cellulose as the base and may contain ingredients contained in the carrier described below other than the hydrophilic ointment base.

Yet another preferred pharmaceutically acceptable topical carrier may include a solution of an acrylic copolymer in a non-aqueous solvent system, which mainly contains polyethylene glycol such as methoxy polyethylene glycol 550 (MPEG). A particular preferred MPEG is SENTRY CARBOWAX MPEG 550 sold by Union Carbide, which is a food/pharmaceutical/cosmetic grade material. Polyethylene glycols are generally non-toxic, water-soluble polymers that are fully biodegradable. In the solution, the acrylic copolymer would preferably be present in a concentration range of 3-6% by weight. Preferably, the acrylic copolymer has a molecular weight of more than 20,000. More preferably, the acrylic copolymer has a molecular weight of more than 100,000 so that it will not be systematically absorbed by the human body or skin. Components of the carrier material described below, other than the hydrophilic ointment base may also be employed in this carrier material.

Suitable hydrophilic ointment bases are known to persons skilled in the art. Exemplary hydrophilic ointment bases suitable for use in the present invention are non-U.S.P. hydrophilic ointment bases such as those made by Fougera, Inc. Sufficient hydrophilic ointment base is employed to act as a topical carrier for the active or non-carrier ingredients of the topical composition. Typically, the hydrophilic ointment base will make up more than about 80% of the total composition, and more preferably about 80-95% of the composition is the hydrophilic ointment base. The hydrophilic ointment base functions as a topical carrier and enhances penetration into the skin. Similar proportions of the hydroxymethyl cellulose-based carrier or acrylic copolymer solution based carrier may also be employed.

The panthenol or panthenol derivatives useful in the present invention include at least D-panthenol, DL-panthenol and mixtures thereof. This component of the topical carrier has skin moisturizing properties and acts as a quick, deep penetrating component of the topical carrier that helps deliver the non-carrier ingredients through the skin to the area to be treated and may also impart a healing effect to damaged tissue. The amount of panthenol or panthenol derivative to be employed is from about 0.25 to about 10 weight percent, more preferably from about 0.5 to about 5 weight percent and most preferably from about 1 to about 2 weight percent, based on the total weight of the topical composition.

The topical carrier of the present invention may also include additional ingredients such as other carriers, moisturizers, humectants, emollients, dispersants, radiation blocking compounds, particularly UV-blockers, as well as other suitable materials that do not have a significant adverse effect on the activity of the topical composition. Preferred additional ingredients for inclusion in the topical carrier are sodium acid phosphate moisturizer, witch hazel extract, glycerine humectant, apricot kernal oil emollient, and corn oil dispersant.

The topical composition of the present invention may also be employed to facilitate wound healing, for the treatment of skin cancer and/or one or more symptoms thereof, or as a topical composition for protecting skin from the harmful effects of radiation, such as radiation breakdown or radiation recall dermatitis.

The topical composition of the present invention is preferably made by cold compounding. This may be an important feature of the invention if one or more of the compounds employed in the topical composition are sensitive to heat or other types of energy in which case the activity of the topical composition may be detrimentally affected as a result of the formulation of the topical compositions in another manner. Thus, the ingredients of the topical composition the present invention are preferably mixed together, without heating using a sufficient amount of the topical carrier to provide a substantially homogeneous cream or ointment. It may be necessary to dissolve, disperse or suspend one or more of the ingredients prior to cold compounding in order to ensure substantially homogeneous distribution of the non-carrier or active ingredients in the topical composition.

A preferred pharmaceutically acceptable topical carrier of the invention can be made using the following ingredients, all based on use of one pound of hydrophilic ointment base. 25-35 parts of a 50% aqueous solution of sodium acid phosphate moisturizing agent, 5-10 parts of D- or DL-panthenol, 5-10 parts of glycerine, 1-3 parts of apricot kernal oil and 10-20 parts of witch hazel extract. For a topical composition of the present invention, particularly preferred combinations of antioxidants, a flavonoid and a compound which regulates cell differentiation and/or cell proliferation for use in the present invention comprises or consists especially of 2-9 parts of a dispersion of vitamins A and D₃ in a corn oil base, 1-4 parts or quercetin, 1-4 parts of vitamin E acetate, 2-4 parts of ascorbyl palmitate and 0.25-2 parts of α-lipoic acid. Optionally, one or more of the optionally ingredients of the topical composition such as glycerin, witch hazel extract, vitamins A and E and/or the ascorbyl palmitate can be reduced or eliminated from a particular topical composition, if desirable, or larger amounts of one type of component, i.e. an antioxidant, can be employed while reducing the amount of another component of the same type or having a similar activity.

When the composition of the present invention is formulated into a topical composition, preferably, the vitamins A and D₃ used in the composition of the present invention may be formulated in a single corn oil dispersion. Generally, every cubic centimeter (cc) of the corn oil dispersion of vitamins A and D₃ used in the present invention may contain about 500,000 to about 2,000,000 IU of vitamin A and about 50,000 to about 200,000 IU of vitamin D₃. Preferably, every cc of the corn oil dispersion of vitamins A and D₃ used in the present invention may contain about 1,000,000 IU of vitamin A and about 100,000 IU of vitamin D₃.

In one preferred embodiment in order to formulate the compound that regulates cell differentiation and/or cell proliferation in the composition of the present invention, which may be administered to a patient topically, it may be advantageous to use a dispersant. Suitable dispersants are known to persons skilled in the art. A particularly suitable dispersant for the compound that regulates cell differentiation and/or cell proliferation is corn oil. Corn oil also has the advantage that it is a natural product. The amount of corn oil used is an amount sufficient to disperse the compound that regulates cell differentiation and/or cell proliferation.

When the composition is formulated into a topical composition, the antioxidant enzyme used in the present invention is preferably skin-absorbable. However, due to its solubility characteristics, vitamin A may need to be formulated in a suitable dispersant such as corn oil in much the same manner as vitamin D₃ as described above when the composition is formulated into a topical composition.

The invention will now be further illustrated by the following example.

### EXAMPLE 1

An oral composition of the present invention is described in Table 1 below. These ingredients may be mixed with a suitable amount of a pharmaceutically acceptable oral carrier described as above to form, for example, tablets for oral administration.

TABLE 1

| Ingredient | Amount Employed | |
|---|---|---|
| Vitamin A palmitate | 10,000 | IU of Vitamin A |
| Vitamin D₃ | 400 | IU |
| β-Carotene | 15,000 | IU |
| Vitamin E | 400 | IU |
| α-Lipoic acid | 150 | mg |
| Quercetin | 1200 | mg |
| Ascorbyl palmitate | 500 | mg |
| Curcumin | 15 | mg |
| Green tea (C&P) | 20 | mg |
| Chlorophyllin | 200 | mg |
| Carboxy ethyl sesquioxide of germanium | 100 | mg |
| Superoxide dismutase | 1,125 | mcg |

This oral composition can be administered 1-5 times daily for the prevention, reduction or treatment of radiation injury prior to, during or after radiation exposure.

### EXAMPLE 2

A topical composition including a mixture of an hydrophilic ointment base, sodium acid phosphate moisturizing agent, a witch hazel extract carrier, glycerine, apricot kernal oil and DL-panthenol, as the pharmaceutically acceptable carrier and vitamins A and D₃, ascorbyl palmitate, α-lipoic acid and vitamin E acetate as the active ingredients which have antioxidant properties and/or regulate cell differentiation and/or cell proliferation was prepared by cold compounding. The formulation of the topical composition is given in Table 2.

The topical composition was prepared by first placing the hydrophilic ointment base in a stainless steel bowl and mixing briskly until the ointment becomes creamy. Then, the sodium acid phosphate, panthenol, ascorbyl palmitate, glycerine, apricot kernal oil, vitamins A and D₃, quercetin, witch hazel extract, vitamin E acetate and α-lipoic acid were added in that order. After each ingredient was added, mixing was continued until all traces of dry ingredients disappeared and a substantially homogeneous mixture was obtained. The final color should be a consistent yellow and the cream should have the consistency of cake frosting. The mixture was then placed in a sterile container. All containers which contact the topical composition during mixing must also be sterilized with, for example, zephiran chloride or a Clorox solution such as betadine.

This composition was topically administered, under the supervision of a physician, to several patients a day before undergoing radiation therapy treatment. The administration of the topical composition was carried out by applying a thin film of the composition to the areas of the skin to be exposed to radiation. The topical composition was applied three times during that day in the morning, noon and at bedtime. All of the patients administered with the topical composition of the present invention experienced much less severe radiation dermatitis after radiation therapy than patients who were not treated with the topical composition of the invention. The effects noted by the patients included reductions in burning, irritation and redness in the areas of skin that were treated. This topical composition can also be administered in a combined treatment involving the oral administration of the composition of Example 1.

TABLE 2

| Ingredient | Amount Employed | |
|---|---|---|
| Hydrophilic ointment base | 1 | pound |
| 50% aqueous solution of Sodium acid phosphate | 25 | cc |
| DL-panthenol | 5 | cc |
| Glycerine | 5 | cc |
| Apricot kernal oil | 3 | cc |
| Witch hazel extract | 12 | cc |
| α-Lipoic acid | 500 | mg |
| Vitamin E acetate | 2 | cc |
| Vitamin A and D₃ dispersion in corn oil | 6 | cc |
| Ascorbyl Palmitate | 2 | grams |
| Quercetin | 2 | grams |

### EXAMPLE 3

Tables 3-7 below exemplify some alternative topical formulations, which may be employed in the method of the present invention without listing all of the ingredients in the pharmaceutically acceptable topical carrier. These alternative formulations may be prepared using the same procedure as described in Example 1.

TABLE 3

| Ingredient | Amount Employed | |
|---|---|---|
| Ascorbyl Palmitate | 2 | grams |
| Hesperidine | 2 | grams |
| Rutin | 2 | grams |
| Vitamin A and D₃ dispersion in corn oil | 3 | cc |
| Vitamin E Acetate | 1 | cc |
| DL Panthenol | 5 | cc |

TABLE 4

| Ingredient | Amount Employed | |
|---|---|---|
| Ascorbyl Palmitate | 2 | grams |
| Ascorbyl Glucosamine | 1 | gram |
| Luteolin | 4 | grams |
| Vitamin A and D₃ dispersion in corn oil | 3 | cc |
| Vitamin E acetate | 1 | cc |
| DL Panthenol | 5 | cc |

TABLE 5

| Ingredient | Amount Employed | |
|---|---|---|
| Ascorbyl Glucosamine | 2 | grams |
| Apigenin | 4 | grams |
| Vitamin A and D₃ dispersion in corn oil | 3 | cc |
| Vitamin E acetate | 1 | cc |
| DL Panthenol | 5 | cc |

TABLE 6

| Ingredient | Amount Employed | |
|---|---|---|
| Ascorbyl Palmitate | 2 | grams |
| γ-Linolenic acid | 500 | mg |
| Rutin | 4 | grams |
| Vitamin A and D₃ dispersion in corn oil | 3 | cc |
| Vitamin E acetate | 1 | cc |
| DL Panthenol | 5 | cc |

TABLE 7

| Ingredient | Amount Employed | |
|---|---|---|
| Ascorbyl Palmitate | 4 | grams |
| Quercetin | 2 | grams |
| Coenzyme Q10 | 500 | mg |
| α-Lipoic acid | 50 | mg |
| Vitamin A and D₃ dispersion in corn oil | 3 | cc |
| Vitamin E acetate | 1 | cc |
| DL Panthenol | 5 | cc |

The foregoing detailed description of the invention and examples are not intended to limit the scope of the invention in any way and should not be construed as limiting the scope of the invention. The scope of the invention is to be determined from the claims appended hereto.

What is claimed is:

1. A method for the prevention, reduction or treatment of radiation injury comprising the step of orally administering to a human prior to expected exposure to radiation, during exposure to radiation or after exposure to radiation, a composition which comprises an amount of one or more compounds effective to regulate at least one of cell differentiation and cell proliferation which is effective, when administered orally, to regulate at least one of cell differentiation and cell proliferation, and an effective amount of one or more antioxidants, wherein the radiation injury being treated is caused by ionizing radiation.

2. A method as claimed in claim 1, wherein the compound that regulates at least one of cell differentiation and cell proliferation is selected from the group consisting of vitamin D₃, 1(S), 3(R)-dihydroxy-20(R)-(1-ethoxy-5-ethyl-5-hydroxy-2-heptyn-1-yl)-9,10-seco-pregna-5(Z), 7(E), 10 (19)-triene, compounds that may be converted or metabolized into vitamin D₃ in the human body, metabolites thereof, and pharmaceutically acceptable salts thereof.

3. A method as claimed in claim 1, wherein the one or more compounds that regulate at least one of cell differentiation and cell proliferation are selected from the group consisting of: cholesterols, 7-dehydrocholestrol, vitamin D₃, 1,25-dihydroxyvitamin D₃, 1(S), 3(R)-dihydroxy-20(R)-(1-ethoxy-5-ethyl-5-hydroxy-2-heptyn-1-yl)-9,10-seco-pregna-5(Z), 7(E), 10 (19)-triene, and 25-hydroxycholecalciferol, calcitriol, and pharmaceutically acceptable salts thereof.

4. A method as claimed in claim 1, wherein the one or more antioxidants are selected from the group consisting of: ascorbyl palmitate, L-ascorbic acid, dehydroascorbic acid, L-threonic acid, L-xylonic acid, L-lyxonic acid and the edible salts of L-threonic acid, L-xylonic acid and L-lyxonic acid, vitamin A, vitamin A ester, vitamin E, vitamin E ester, α-lipoic acid carotenoid, chlorophyllin, chlorophyllin salt, coenzyme Q10, glutathione, green tea polyphenol, galangin, rutin, luteolin, morin, fisetin, silymarin, apigenin, gingkolides, hesperitin, cyanidin, citrin, curcuminoid, and pharmaceutically acceptable salts thereof.

5. A method as claimed in claim 1, wherein the compound that regulates at least one of cell differentiation and cell proliferation comprises vitamin D₃, and the antioxidant comprises ascorbyl palmitate, curcumin, vitamin A palmitate, vitamin E, α-lipoic acid, green tea polyphenol, and chlorophyllin.

6. A method as claimed in claim 1 wherein the antioxidant comprises one or more antioxidant enzymes.

7. A method as claimed in claim 1, wherein the composition further comprises at least one compound selected from the group consisting of: flavonoids and flavonoid derivatives.

8. A method as claimed in claim 7, wherein the flavonoids and flavonoid derivatives are selected from the group consisting of: 1,2,3,6-tetra-o-gallyol-β-d-glucose; 2′o-acetylacetoside; 3,3′,4-tri-o-methyl-ellagic acid; 6,3′,4′-trihydroxy-5,7,8-trimethoxyflavone; 6-hydroxy-luteolin; 6-hydroxykaempferol-3,6-dimethyl ether; 7-o-acetyl-8-epi-loganic acid; acacetin; acetoside; acetyl trisulfate quercetin; amentoflavone; apigenin; apiin; astragalin; avicularin; axillarin; baicalein; brazilin; brevifolin carboxylic acid; caryophyllene; chrysin-5,7-dihydroxyflavone; chrysoeriol; chrysosplenol; chrysosplenoside-a; chrysosplenoside-d; cosmosiin; δ-cadinene; dimethylmussaenoside; diacerylcirsimaritin; diosmetin; dosmetin; ellagic acid; ebinin; ethyl brevifolin carboxylate; flavocannibiside; flavosativaside; genistein; gossypetin-8-glucoside; haematoxylin; hesperidine; hispiduloside; hyperin; indole; iridine; isoliquiritigenin; isoliquiritin; isoquercitrin; jionoside; juglanin; kaempferoi-3-rhamnoside; kaempferol-3-neohesperidoside; kolaviron; licuraside; linariin; linarin; lonicerin; luteolin; luetolin-7-glucoside; luteolin-7-glucoside; luetolin-7-glucoronide; macrocarpal-a; macrocarpal-b; macrocarpal-d; macrocarpal-g; maniflavone; methy scutellarein; naringenin; naringin; nelumboside; nepetin; nepetrin; nerolidol; oxyayanin-a; pectolinarigenin; pectolinarin; quercetagetin; quercetin; quercimertrin; quercitrin; quercitryl-2″ acetate; reynoutrin; rhamnetin; rhoifolin; rutin; scutellarein; sideritoflavone; sophoricoside; sorbarin; spiraeoside; trifolin; vitexin; and wogonin.

9. A method as claimed in claim 7, wherein the flavonoids and flavonoid derivatives are selected from the group consisting of: quercetin, quercetrin, myricetin, kaempferol and myrecetrin.

10. A method as claimed in claim 1, wherein the composition further comprises one or more ingredients selected from the group consisting of selenium and selenium compounds.

11. A method as claimed in claim 1, wherein the composition further comprises one or more ingredients selected from the group consisting of organic germanium, Korean ginseng, an extract of Korean ginseng, American ginseng, an extract of American ginseng, Siberian ginseng and an extract of Siberian ginseng.

12. A method as claimed in claim 1, wherein the composition further comprises one or more ingredients selected from the group consisting of anti-inflammatories, and B-complex vitamins.

13. A method as claimed in claim 1, wherein a ratio of the amount of the compound that regulates at least one of cell differentiation and cell proliferation to the amount of antioxidant from about 200 IU per gram of antioxidant to about 3 million IU per gram of antioxidant.

14. A method as claimed in claim 1, wherein a ratio of the amount of the compound that regulates at least one of cell differentiation and cell proliferation to the amount of antioxidant is from about 1800 IU per gram of antioxidant to about 1 million IU per gram of antioxidant.

15. A method as claimed in claim 1, wherein a ratio of the amount of the compound that regulates at least one of cell differentiation and cell proliferation to the amount of antioxidant is from about 5000 IU per gram of antioxidant to about 200,000 IU per gram of antioxidant.

16. A method as claimed in claim 1, wherein the ionizing radiation is selected from the group consisting of alpha radiation, beta radiation, gamma radiation and x-ray radiation.

17. An oral composition for preventing, reducing or treating radiation injury comprising:
an amount of one or more compounds effective to regulate at least one of cell differentiation and cell proliferation which is effective, when administered orally, to regulate at least one of cell differentiation and cell proliferation, an effective amount of one or more antioxidants, and at least one of a pharmaceutically acceptable carrier for an oral composition.

18. A composition as claimed in claim 17, wherein the compound that regulates at least one of cell differentiation and cell proliferation is selected from the group consisting of vitamin D₃, 1(S), 3(R)-dihydroxy-20(R)-(1-ethoxy-5-ethyl-5-hydroxy-2-heptyn-1-yl)-9,10-seco-pregna-5(Z), 7(E), 10 (19)-triene, compounds that may be converted or metabolized into vitamin D₃ in the human body, metabolites thereof, and pharmaceutically acceptable salts thereof.

19. A composition as claimed in claim 17, wherein the one or more compounds that regulate at least one of cell differentiation and cell proliferation are selected from the group consisting of: cholesterols, 7-dehydrocholestrol, vitamin D₃, 1,25-dihydroxyvitamin D₃, 1(S), 3(R)-dihydroxy-20(R)-(1-ethoxy-5-ethyl-5-hydroxy-2-heptyn-1-yl)-9,10-seco-pregna-5(Z), 7(E), 10 (19)-triene, and 25-hydroxycholecalciferol, calcitriol, and pharmaceutically acceptable salts thereof.

20. A composition as claimed in claim 17, wherein the one or more antioxidants are selected from the group consisting of: ascorbyl palinitate, L-ascorbic acid, dehydroascorbic acid, L-threonic acid, L-xylonic acid, L-lyxonic acid and the edible salts of L-threonic acid, L-xylonic acid and L-lyxonic acid, vitamin A, vitamin A ester, vitamin E, vitamin E ester, α-lipoic acid carotenoid, chlorophyllin, chlorophyllin salt, coenzyme Q10, glutathione, green tea polyphenol, galangin, rutin, luteolin, morin, fisetin, silymarin, apigenin, gingkolides, hesperitin, cyanidin, citrin, curcuminoid, and pharmaceutically acceptable salts thereof.

21. A composition as claimed in claim 17, wherein the compound that regulates at least one of cell differentiation and cell proliferation comprises vitamin D₃, and the antioxidant comprises ascorbyl palmitate, curcumin, vitamin A palmitate, vitamin E, α-lipoic acid, green tea polyphenol, and chlorophyllin.

22. A composition as claimed in claim 17, wherein the antioxidant comprises one or more antioxidant enzymes.

23. A composition as claimed in claim 17, wherein the composition further comprises at least one compound selected from the group consisting of: flavonoids and flavonoid derivatives.

24. A composition as claimed in claim 23, wherein the flavonoids and flavonoid derivatives are selected from the group consisting of: 1,2,3,6-tetra-o-gallyol-β-d-glucose; 2′o-acetylacetoside; 3,3′,4-tri-o-methyl-ellagic acid; 6,3′,4′-trihydroxy-5,7,8-trimethoxyflavone; 6-hydroxy-luteolin; 6-hydroxykaempferol-3,6-dimethyl ether; 7-o-acetyl8-epi-loganic acid; acacetin; acetoside; acetyl trisulfate quercetin; amentoflavone; apigenin; apiin; astragalin; avicularin; axillarin; baicalein; brazilin; brevifolin carboxylic acid; caryophyllene; chrysin-5,7-dihydroxyflavone; chrysoeriol; chrysosplenol; chrysosplenoside-a; chrysosplenoside-d; cosmosiin; δ-cadinene; dimethylmussaenoside; diacerylcirsimaritin; diosmetin; dosmetin; ellagic acid; ebinin; ethyl brevifolin carboxylate; flavocannibiside; flavosativaside; genistein; gossypetin-8-glucoside; haematoxylin; hesperidine; hispiduloside; hyperin; indole; iridine; isoliquiritigenin; isoliquiritin; isoquercitrin; jionoside; juglanin; kaempferol-3-rhamnoside; kaempferol-3-neohesperidoside; kolaviron; licuraside; linariin; linarin; lonicerin; luteolin; luetolin-7-glucoside; luteolin-7-glucoside; luetolin-7-glucoronide; macrocarpal-a; macrocarpal-b; macrocarpal-d; macrocarpal-g; maniflavone; methy scutellarein; naringenin; naringin; nelumboside; nepetin; nepetrin; nerolidol; oxyayanin-a; pectolinarigenin; pectolinarin; quercetagetin; quercetin; quercimertrin; quercitrin; quercitryl-2″acetate; reynoutrin; rhamnetin; rhoifolin; rutin; scutellarein; sideritoflavone; sophoricoside; sorbarin; spiraeoside; trifolin; vitexin; and wogonin.

25. A composition as claimed in claim 23, wherein the flavonoids and flavonoid derivatives are selected from the group consisting of: quercetin, quercetrin, myricetin, kaempferol and myrecetrin.

26. A composition as claimed in claim 17, wherein the composition further comprises one or more ingredients selected from the group consisting of selenium and selenium compounds.

27. A composition as claimed in claim 17, wherein the composition further comprises one or more ingredients selected from the group consisting of organic germanium, Korean ginseng, an extract of Korean ginseng, American ginseng, an extract of American ginseng, Siberian ginseng and an extract of Siberian ginseng.

28. A composition as claimed in claim 17, wherein the composition further comprises one or more ingredients selected from the group consisting of anti-inflammatories, and B-complex vitamins.

29. A composition as claimed in claim 17, wherein the composition is in a form selected from the group consisting of tablets, capsules, lozenges, troches, hard candies, powders, sprays, elixirs, syrups, suspensions, solutions, mouthwashes, sprays, liquids, soft candy, gum drops, liquid filled candies, chewing gum bases, toothpastes, nasal aerosols or inhalants.

30. A composition as claimed in claim 17, wherein a ratio of the amount of the compound that regulates at least one of cell differentiation and cell proliferation to the amount of antioxidant from about 200 IU per gram of antioxidant to about 3 million IU per gram of antioxidant.

31. A composition as claimed in claim 17, wherein a ratio of the amount of the compound that regulates at least one of cell differentiation and cell proliferation to the amount of antioxidant is from about 1800 IU per gram of antioxidant to about 1 million IU per gram of antioxidant.

32. A composition as claimed in claim 17, wherein a ratio of the amount of the compound that regulates at least one of cell differentiation and cell proliferation to the amount of antioxidant is from about 5000 IU per gram of antioxidant to about 200,000 IU per gram of antioxidant.

33. An oral composition for preventing, reducing or treating radiation injury comprising:
non-carrier ingredients, and
a pharmaceutically acceptable oral carrier for an oral composition,
wherein every gram of non-carrier ingredients in the oral composition comprises 3,800-4,800 IU of vitamin A palmitate; 2,400-7,200 IU of beta carotene; 240-480 IU vitamin D₃; 95-300 IU of vitamin E in a form of alpha-tocopherol; 48-72 mg of alpha-lipoic acid; 280-580 mg of quercetin, 120-240 mg of ascorbyl palmitate; 4.5-7.2 mg of curcumin; 4.5-10 mg of green tea (C&P); 45-100 mg of chlorophyllin; 24-100 mg of carboxy ethyl sesquioxide of germanium and 180-540 mcg of superoxide dismutase.

34. An oral composition as claimed in claim 33, where the non-carrier ingredients comprise one or more compounds selected from the group consisting of: ascorbyl palmitate, L-ascorbic acid, dehydroascorbic acid, L-threonic acid, L-xylonic acid, L-lyxonic acid and the edible salts of L-threonic acid, L-xylonic acid and L-lyxonic acid.

35. An oral composition as claimed in claim 34, further comprising:
beta carotene; curcumin; green tea polyphenol; chlorophyllin; and an antioxidant enzyme.

36. An oral composition as claimed in claim 34, wherein the vitamin A is in a form of a vitamin A palmitate, and the antioxidant enzyme is superoxide dismutase.

37. An oral composition as claimed in claim 34, wherein a ratio of the amount of the compound that regulates at least one of cell differentiation and cell proliferation to the amount of antioxidant from about 200 IU per gram of antioxidant to about 3 million IU per gram of antioxidant.

38. An oral composition as claimed in claim 34, wherein a ratio of the amount of the compound that regulates at least one of cell differentiation and cell proliferation to the amount of antioxidant is from about 1800 IU per gram of antioxidant to about 1 million IU per gram of antioxidant.

39. An oral composition as claimed in claim 34, wherein a ratio of the amount of the compound that regulates at least one of cell differentiation and cell proliferation to the amount of antioxidant is from about 5000 IU per gram of antioxidant to about 200,000 IU per gram of antioxidant.
